(19) United States
(12) Patent Application Publication
(10) Pub. No.: US 2009/0275634 A1
(43) Pub. Date: Nov. 5, 2009

(54) ANTISENSE OLIGONUCLEOTIDES AGAINST ACETYLCHOLINESTERASE FOR TREATING INFLAMMATORY DISEASES

(75) Inventors: Eli Hazum, Tel Aviv (IL); Lior Carmon, Tel Aviv (IL)

Correspondence Address: K&L Gates LLP, P.O. Box 1135, CHICAGO, IL, 60690, US

(73) Assignee: AMARIN CORPORATION PLC, Dublin (IE)

(21) Appl. No.: 12/296,455

(22) PCT Filed: Mar. 29, 2007
(86) PCT No.: PCT/IL07/00413

§ 371 Date: Oct. 8, 2008

(30) Foreign Application Priority Data
Apr. 10, 2006 (US) 60790546

(51) Int. Cl. A61K 31/7052; A61P 29/00

(52) U.S. Cl. 514/44 A; 514/44 R

(57) ABSTRACT

The present invention relates to novel uses of antisense oligonucleotides targeted to the coding region of acetylcholinesterase (AChE) for treating inflammatory disorders other than inflammatory disorders of the central nervous system or the peripheral nervous system innervating voluntary muscles. More particularly, the present invention relates to uses of antisense oligodexoynucleotides targeted to AChE mRNA for treating inflammatory disease of the gastrointestinal tract including inflammatory bowel disease.

### FIELD OF THE INVENTION

The present invention relates to novel uses of antisense oligonucleotides targeted to the coding region of acetylcholinesterase (AChE) for treating inflammatory disorders other than inflammatory disorders of the central nervous system or the peripheral nervous system innervating voluntary muscles. More particularly, the present invention relates to uses of antisense oligodexoynucleotides targeted to ACHE mRNA for treating inflammatory diseases of the gastrointestinal tract including inflammatory bowel disease.

### BACKGROUND OF THE INVENTION

Inflammatory processes play a crucial role in defense against pathogen invaders as well as in healing and recovery following various types of injury. The magnitude and duration of inflammatory responses have to be tightly regulated, as excessive inflammatory responses can be detrimental, leading to autoimmune diseases, neurodegeneration, sepsis, trauma and other pathological conditions.

It has long been recognized that regulation of inflammatory processes is mediated both by immune responses (particularly the secretion of anti-inflammatory cytokines) and by neuroendocrine factors, particularly by glucocorticoids. Recently it became evident that neural mechanisms are also involved in limiting inflammatory responses. In particular, it was found that cholinergic neurons inhibit acute inflammation, providing a rapid, localized, and adaptive anti-inflammatory reflex system. In the periphery, acetylcholine (ACh) is mainly released by the efferent vagus nerve. It significantly attenuates the production of the pro-inflammatory cytokines TNFA, interleukin-1β (IL-1β), IL-6 and IL-18, but not the anti-inflammatory cytokine IL-10. It was also shown that IL-1 causes acetylcholinesterase (ACHE) over-production both in PC12 cells and in the rat cortex, suggesting a closed loop whereby ACh suppresses IL-1 production, thus ablating the induction of AChE production.

Numerous diseases are believed to result from autoimmune or inflammatory mechanisms. Prominent among these are Crohn's disease or inflammatory bowel disease.

Crohn's disease (CD) is a chronic inflammatory disease of the gastrointestinal tract having unclear etiology. It primarily causes ulcerations of the small and large intestines, but can affect the digestive system anywhere from the mouth to the anus. Various terms are used to describe CD, and tend to reflect the portion of the gastrointestinal tract affected. Involvement of the large intestine (colon) only has been termed Crohn's colitis or granulomatous colitis, while involvement of the small intestine only has been termed Crohn's enteritis. Disease in the terminal portion of the small intestine i.e., the ileum, has been termed Crohn's ileitis. When both the small intestine and the large intestine are involved, the condition has been termed Crohn's enterocolitis or ileocolitis.

CD is related closely to ulcerative colitis, another chronic inflammatory condition that involves only the colon. Together, CD and ulcerative colitis are frequently referred to as inflammatory bowel disease (IBD). Ulcerative colitis and CD have no medical cure, and once the diseases are manifest, they tend to fluctuate between periods of remission and relapse. Together, these conditions affect approximately 500,000 to 2 million people in the United States.

The development of CD is likely multi-factorial, but appears to involve a dysregulated immune response to pathogenic and/or resident normal bacteria in a genetically pre-disposed host.

Close association of bacteria with the intestinal epithelial surface and/or penetration of the mucosal barrier trigger a cascade of signaling events in CD. This includes increased production of cytokines including TNFα, IL-8, GROα, MCP-1, cyclooxygenase, prostaglandins E2 and F2α, nitric oxide synthase and increased surface expression of the adhesion molecule ICAM-1. Up-regulated cytokine expression is mediated by a common signal transduction pathway involving NF-κB. Increased local concentrations of cytokines initiate the biochemical cascade which produces tissue injury.

Current IBD drug therapies are inadequate. The two most widely used drug families are steroids and 5-aminosalicylic acid (5-ASA) drugs, both of which reduce inflammation of the affected parts of the intestines. Immunosuppressive drugs such as 6-mercaptopurine are increasingly used for long-term treatment of IBD. They are particularly used for patients dependent on chronic high-dose steroid therapy with its severe and predictable side effects. Other medications include antibodies against proinflammatory cytokines such as IL-6 and TNF or antibiotics.

As these medications have many side effects and have not been successful in curtailing the disease, there has been an urgent need to develop satisfactory treatment of IBD.

International Publication No. WO 03/002739 discloses an antisense oligonucleotide designated hEN101 targeted to the coding region of the human AChE, which selectively suppresses the expression of AChE-R isoform of the enzyme. WO 03/002739 further discloses and claims pharmaceutical composition comprising hEN101 for the treatment or prevention of a progressive neuromuscular disorder, wherein said disorder involves muscle distortion, muscle re-innervation or neuromuscular junction abnormalities. Among the disorders to be treated with EN101, myasthenia gravis, Eaton-Lambert disease, muscular dystrophy, amyotrophic lateral sclerosis, and multiple sclerosis are disclosed. These therapeutic targets are based on prevention of loss or retention of acetylcholine pathways for neuromuscular functions. Specific examples are provided to demonstrate the efficacy of hEN101 in reducing myasthenia gravis severity in experimental autoimmune myasthenic gravis (EAMG) rats.

International Publication No. WO 2005/039480 discloses the use of an inhibitor of AChE expression as an anti-inflammatory agent and as a suppressor of pro-inflammatory cytokine release. WO 2005/039480 further discloses pharmaceutical compositions comprising an inhibitor of AChE expression for the treatment or prevention of inflammation in the joints, central nervous system, gastrointestinal tract, endocardium, pericardium, lung, eyes, skin and urogenital system. Specific examples are provided to demonstrate the ability of hEN101 to suppress neuronal pro-inflammatory cytokines in hEN101-treated monkeys. However, no specific enablement or guidance is provided for the use of EN101 in the treatment of inflammatory disorders which are not associated with the central nervous system (CNS) or peripheral nervous system (PNS) innervating voluntary muscles.

Treatment of experimental autoimmune encephalomyelitis (EAE), a CNS inflammatory disease, by EN101 was recently disclosed by Nizri et al. (Neuropharmacol. 2006, 50: 540-547). The results of Nizri's study raised the importance of cholinergic balance in CNS inflammatory disorders such as multiple sclerosis as well as in neurological disorders such as Alzheimer's disease and myasthenia gravis.

There is thus an unmet need for effective and safe methods for treating patients suffering from inflammatory disorders which are not associated with the CNS or PNS.

### SUMMARY OF THE INVENTION

The present invention provides methods for treating inflammatory disorders other than inflammatory disorders of the central nervous system or the peripheral nervous system innervating the voluntary muscles, the methods comprise administering to a subject in need thereof a pharmaceutical composition comprising as an active agent an antisense oligonucleotide targeted to acetylcholinesterase (AChE) mRNA.

The present invention is based on the unexpected discovery that administration of an antisense oligodeoxynucleotide designated EN101 targeted to the mRNA of human AChE readthrough isoform having the nucleotide sequence 5′-CTGCCACGTTCTCCTGCACC-3′ set forth in SEQ ID NO:1, and particularly, a nuclease resistant form of EN101 having the nucleotide sequence 5′-CTGCCACGTTCTCCTGCA*C*C*-3′ set forth in SEQ ID NO:2 which includes three 3′ terminal 2-O-methyl groups marked by (*), lead to amelioration of the symptoms associated with inflammatory bowel disease (IBD) in an animal model. The efficacy of EN101 in ameliorating the symptoms associated with IBD is highly significant and comparable to that achieved by dexamethasone. EN101 was found to exert its therapeutic effects at low doses and therefore it is of high advantage in treating chronic inflammatory disorders, particularly inflammatory gastrointestinal disorders.

While EN101 was previously shown to be efficient in treating neuromuscular or neuronal degenerative disorders such as myasthenia gravis or multiple sclerosis, it is now disclosed for the first time that EN101 is efficient in treating inflammatory disorders which are not associated with the central nervous system (CNS) or with neuronal or neuromuscular degeneration. EN101 is shown to be particularly useful in treating gastrointestinal disorders.

It is to be understood that the etiology or pathogenesis of inflammatory gastrointestinal disorders is unresolved. Though some theories suggest that stress or allergy-induced bacteria elicit these diseases, and therefore steroids or antibiotics are indicated for treating these disorders, the present invention surprisingly discloses that an antisense oligonucleotide targeted to ACHE mRNA can be used as a prevailing medication for gastrointestinal disorders.

According to the first aspect, the present invention provides a method for treating an inflammatory disorder comprising administering to a subject in need thereof a therapeutically effective amount of a pharmaceutical composition comprising as an active agent an antisense oligonucleotide targeted to ACHE mRNA and a pharmaceutically acceptable carrier, wherein the inflammatory disorder is other than an inflammatory disorder of the central nervous system (CNS) or the peripheral nervous system innervating the voluntary muscles.

According to some embodiments, the subject is a mammal. According to still further embodiments, the mammal is a human.

According to further embodiments, the inflammatory disorder is an inflammatory gastrointestinal disorder. According to yet further embodiments, the inflammatory gastrointestinal disorder is selected from the group consisting of chronic inflammatory gastrointestinal disorders and acute inflammatory gastrointestinal disorders. According to yet further embodiments, the chronic inflammatory gastrointestinal disorder is inflammatory bowel disease selected from the group consisting of Crohn's disease, Crohn's colitis, Crohn's enteritis, and ulcerative colitis. According to still further embodiments, the inflammatory gastrointestinal disorder is attributed to an immune function disorder. Examples of such immune function disorders include, but are not limited to, acquired immunodeficiency syndrome, chronic granulomatous disease, hypogammaglobulinemia, agammaglobulinemia, leukocyte adhesion deficiency, cyclic neutropenia, glycogen storage disease 1b, celiac disease, infectious gastritis or enterocolitis.

According to further embodiments, the antisense oligonucleotide targeted to ACHE mRNA is an antisense oligodeoxynucleotide having the nucleotide sequence selected from the group consisting of SEQ ID NOs:1, 3 to 5. According to still further embodiments, the antisense oligodeoxynucleotide is nuclease resistant. According to additional embodiments, the nuclease resistant antisense oligonucleotide comprises at least one of the last three nucleotides at the 3′ terminus in a 2-O-methylated form. Preferably, the three last nucleotides at the 3′ terminus of the antisense oligodeoxynucleotide are 2-O-methylated. According to a preferred embodiment, the antisense oligodeoxynucleotide has a nucleotide sequence as set forth in SEQ ID NO:2. According to still further embodiments, the antisense oligodeoxynucleotide comprises at least one phosphorothioate bond. According to further embodiments, the antisense oligodeoxynucleotide comprises a phosphorothioate bond linking the two last nucleotide bases at the 3′-terminus.

According to still further embodiments, administering the pharmaceutical composition is selected from the group consisting of oral, intravenous, intraarterial, intraperitoneal, subcutaneous, transdermal, intramuscular, intranasal, and inhalation administration routs. According to a preferred embodiment, administering the pharmaceutical composition is performed by oral administration.

According to yet further embodiments, the pharmaceutical composition is formulated in a form selected from the group consisting of pellets, tablets, capsules, solutions, suspensions, emulsions, gels, creams, transdermal patches and depots.

According to still further embodiments, the pharmaceutical composition of the invention is administered once daily. According to yet further embodiments, the pharmaceutical composition comprising the antisense oligonucleotide set forth in SEQ ID NO:2 is administered at a dosage of 0.1 mg to 20 mg per day.

According to another aspect, the present invention provides uses of the antisense oligonucleotides targeted to ACHE mRNA according to the principles of the present invention for the preparation of a medicament for treating an inflammatory disorder, wherein the inflammatory disorder is other than an inflammatory disorder of the central nervous system (CNS) or the peripheral nervous system innervating the voluntary muscles.

These and other embodiments of the present invention will be better understood in relation to the figures, description, examples and claims that follow.

### BRIEF DESCRIPTION OF THE FIGURES

FIG. 1 shows the effect of oral administration of EN101 on colitis manifestation. Colitis-induced mice were treated daily with various doses of EN101 beginning one day or two days after colitis induction and for the subsequent seven days. The disease score was evaluated.

FIG. 2 shows the effect of increasing concentrations of EN101 on colitis-induced mice. EN101 was administered daily to colitis-induced mice one day before colitis induction and for the subsequent seven days. The disease score was evaluated.

FIG. 3 shows the effect of high doses of EN101 on colitis-induced mice. EN101 at 100, 200 or 500 mg/Kg was administered daily to colitis-induced mice one day before colitis induction and for the subsequent seven days. The disease score was evaluated.

FIGS. 4A-G show photomicrographs of colon sections obtained from colitis-induced mice. FIGS. 4A-C show micrographs of colon sections obtained from untreated colitis-induced mice. FIGS. 4D and 4E show micrographs of a colon section obtained from dexamethasone treated colitis-induced mice. FIGS. 4F and 4G show micrographs of colon sections obtained from EN101 treated colitis-induced mice.

FIGS. 5A-B show FACS analysis of peritoneal macrophages. The number of total peritoneal macrophages was detected by the expression of forward scatter (FCS; FIG. 5A), while the number of activated macrophages was detected by the expression of MAC-1 (FIG. 5B).

FIGS. 6A-B show the total number of peritoneal macrophages (FIG. 6A) or the number of activated macrophages (FIG. 6B) after 72 hrs treatment with either LPS, CpG, a control sequence of CpG, and EN101.

FIGS. 7A-D show the effect of LPS, CpG, a control sequence of CpG, and EN101 on the secretion of IL-6 (FIG. 7A), TNF-α (FIG. 7B), MIP-2 (FIG. 7C), and IL-1α (FIG. 7D) from peritoneal macrophages.

FIGS. 8A-B show the number of MAC-1 peritoneal macrophages derived from wild type mice (C57BL6) (FIG. 8A) or from MyD88 KO mice (FIG. 8B) after treatment with LPS, CpG, and EN101. A control group (CNRL), which was incubated in medium only, is also presented.

FIGS. 9A-D show the level of MIP-2 in the supernatant of peritoneal macrophages derived from wild type mice (C57BL6) (FIG. 9A, B) or from MyD88 KO mice (FIG. 9C, D) after treatment with LPS, CpG, and EN101. A control group (CNRL), which was incubated in medium only, is also presented.

FIGS. 10A-D show the total number of peritoneal macrophages (FIG. 10A-B) or the number of activated macrophages (FIG. 10C-D) after 72 hrs of treatment with CpG (1 or 5 μg/ml) or EN101 (0.1, 1 or 100 μg/ml).

FIGS. 11A-B show the levels of the chemokine MIP-2 in the supernatants of peritoneal macrophages from WT C57BL6 mice (FIG. 11A) or TLR9 KO mice (FIG. 11B) after 24 or 72 hours of treatment with EN101 or CpG.

### DETAILED DESCRIPTION OF THE INVENTION

The present invention relates to new uses of antisense oligonucleotides targeted to acetylcholinesterase (ACHE) mRNA. Particularly, the invention relates to treatment methods which provide symptomatic relief and/or induce remission in patients suffering from inflammatory disorders, particularly inflammatory gastrointestinal disorders.

The term “inflammatory gastrointestinal disorders” as used herein refers to disorders associated with inflammation of the mucosal layer of the gastrointestinal tract, and encompasses acute and chronic inflammatory conditions. Acute inflammation is generally characterized by a short time of onset and infiltration or influx of neutrophils.

The term “chronic inflammatory gastrointestinal disorder” refers to inflammation of the mucosal layer of the gastrointestinal tract which is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic inflammatory gastrointestinal disorder may be expected to require a long period of supervision, observation, or care. Chronic inflammatory gastrointestinal disorders include, but are not limited to, inflammatory bowel disease (IBD), Crohn's disease, and ulcerative colitis.

“Mucosal layer of the gastrointestinal tract” is meant to include mucosa of the bowel (including the small intestine and large intestine), rectum, stomach (gastric) lining, oral cavity, and the like.

As used herein “inflammatory bowel disease” or “IBD” refers to any of a variety of diseases characterized by inflammation of all or part of the intestines. Examples of inflammatory bowel disease include, but are not limited to, Crohn's disease, Crohn's colitis, Crohn's ileitis and ulcerative colitis.

Crohn's disease (also known as regional enteritis or ulcerative ileitis) is a chronic inflammatory disease of unknown etiology which can affect any part of the bowel. The most prominent feature of the disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. The course of the disease may be continuous or relapsing, mild or severe but it is not curable by resection of the involved segment of bowel. Most patients with Crohn's disease require surgery at some point, but subsequent relapse is common and continuous medical treatment is usual.

Ulcerative colitis is a chronic inflammatory disease of unknown etiology afflicting the large intestine. The course of the disease may be continuous or relapsing, mild or severe. The earliest lesion is an inflammatory infiltration with abscess formation at the base of the crypts of Lieberkuhn. Coalescence of these distended and ruptured crypts tends to separate the overlying mucosa from its blood supply, leading to ulceration. Signs and symptoms of the disease include cramping, lower abdominal pain, rectal bleeding, and frequent, loose discharges consisting mainly of blood, pus, and mucus with scanty fecal particles.

Ulcerative colitis can be induced by environmental insults or associated with a therapeutic regimen, such as administration of chemotherapy, radiation therapy, and the like. Colitis can be associated with conditions such as chronic granulomatous disease, celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammatory disorders caused by an infectious agent.

It is to be appreciated that inflammatory gastrointestinal disorders may also be secondary to acquired or inherited immune function disorders, including acquired immunodeficiency syndrome, hypogammaglobulinemia, agammaglobulinemia, leukocyte adhesion deficiency, cyclic neutropenia, and glycogen storage disease 1b.

According to one aspect, the present invention provides a method for treating an inflammatory disorder other than an inflammatory disorder in the CNS or PNS innervating voluntary muscles, the method comprises administering to a subject in need thereof a pharmaceutical composition comprising a therapeutically effective amount of an antisense oligonucleotide targeted to acetylcholinesterase (ACHE) mRNA and a pharmaceutically acceptable carrier.

The present invention employs antisense oligonucleotides for use in modulating the expression of nucleic acid molecules encoding ACHE, ultimately modulating the amount of AChE produced. This is accomplished by providing oligonucleotides which specifically hybridize with mRNA encoding AChE.

This relationship between an antisense oligonucleotide and its complementary nucleic acid target, to which it hybridizes, is commonly referred to as “antisense”.

The antisense oligonucleotides targeted to AChE mRNA are preferably nuclease resistant. Preferably, the antisense oligonucleotides selectively inhibit the ACHE-R mRNA. AChE-R designates the “readthrough” isoform of AChE, which mRNA includes pseudo-intron I4.

Examples of antisense oligonucleotides targeted to ACHE mRNA that can be used for treating inflammatory disorders according to the principles of the present invention have the following sequences:

(SEQ ID NO: 1)

5′-CTGCCACGTTCTCCTGCACC-3′
designated EN101 or human EN101 (hEN101);

(SEQ ID NO: 2)

5′-CTGCCACGTTCTCCTGCA*C*C*-3′
designated nuclease resistant EN101 or hEN101,
wherein the three 3′ terminal residues are
modified with 2-O-methyl groups (*).

(SEQ ID NO: 3)

5′-CTGCAATATTTTCTTGCACC-3′
designated mouse EN101 (mEN101);

(SEQ ID NO: 4)

5′-CTGCCATATTTTCTTGTACC-3′
designated rat EN101 (rEN101);

(SEQ ID NO: 5)

5′-GGGAGAGGAGGAGGAAGAGG-3′
designated hEN103.

It is to be understood that the antisense oligonucleotides of the invention are preferably oligodeoxynucleotides, but ribonucleotides, nucleotide analogs, or mixtures thereof are contemplated by the invention.

Antisense oligonucleotides targeted to AChE mRNA have been described in WO 03/002739, WO 2005/039480, U.S. Pat. No. 7,074,915, and U.S. Patent Publication No. 2006/0069051, the content of which is incorporated by reference as if fully set forth herein. WO 03/002739 discloses the use of the antisense oligonucleotide designated hEN101 for treating myasthenia gravis.

Nuclease resistant antisense oligonucleotides can be prepared by various methods known in the art. Reference is made to International Publication No. WO98/26062, which discloses that oligonucleotides can be made nuclease resistant e.g., by replacing phosphodiester internucleotide bonds with phosphorothioate bonds, replacing the 2′-hydroxy group of one or more nucleotides by 2′-O-methyl groups, fluoridating a nucleotide, or adding a nucleotide sequence capable of forming a loop structure under physiological conditions to the 3′ end of the antisense oligonucleotide sequence.

Thus, in particular embodiments, the nuclease resistant antisense oligodeoxynucleotide of the invention has at least one of the last three 3′ terminus nucleotides as 2′-O-methylated, preferably the last three 3′ terminus nucleotides are 2′-O-methylated. Preferably, the ACHE antisense oligodeoxynucleotide has the nucleotide sequence set forth in SEQ ID NO:2. In alternative embodiments, the nuclease resistant antisense oligodeoxynucleotide of the invention has at least one of the last 3′-terminus nucleotides fluoridated. In still alternative embodiments, the nuclease resistant antisense oligodeoxynucleotide of the invention comprises phosphorothioate bonds linking between at least two of the last 3′-terminus nucleotide bases, preferably phosphorothioate bonds link between the last four 3′-terminal nucleotide bases. In still alternative embodiments, nuclease resistance is achieved by adding a nucleotide sequence capable of forming a loop structure under physiological conditions to the 3′ end of the antisense oligodeoxynucleotide sequence. An example for a loop forming structure is the sequence 5′-CGCGAAGCG-3′ (SEQ ID NO:6), which can be added to the 3′ end of a given antisense oligonucleotide to impart nuclease resistance thereon.

Phosphorothioate-modified oligonucleotides are generally regarded as safe and free of side effects. The antisense oligonucleotides of the present invention have been found to be effective as partially phosphorothioates and yet more effective as partially 2′-O-methyl protected oligonucleotides. WO 98/26062 teaches that AChE antisense oligonucleotides containing three phosphorothioate bonds out of about twenty internucleotide bonds are generally safe to use in concentrations of between about 1 and 10 μM. However, for long-term applications, oligonucleotides that do not release toxic groups when degraded may be preferred. These include 2′-O-methyl protected oligonucleotides, but not phosphorothioate oligonucleotides. A further advantage of 2′-O-methyl protection over phosphorothioate protection is the reduced amount of oligonucleotide that is required for AChE suppression. This difference is thought to be related to the improved stability of the duplexes obtained when the 2′-O-methyl protected oligonucleotides are used (Lesnik, E. A. and Freier, S. M., Biochemistry 37,6991-7, 1998). An alternative explanation for the greater potency of the 2′-O-methyl oligonucleotides is that this modification may facilitate penetration of the oligonucleotide chain through the cell membrane. A further advantage of 2′-O-methyl protection is the better protection against nuclease-mediated degradation that it confers, thus extending the useful life time of antisense oligonucleotides protected in this way.

It is to be appreciated that the pharmaceutical composition of the invention can comprise as an active agent a combination of at least two antisense oligonucleotides as defined in the invention, or functional analogs, derivatives or fragments thereof.

A “fragment” of an oligonucleotide sequence of the present invention is meant to refer to any nucleotide subset of the oligonucleotide capable of inhibiting AChE expression. A “variant” of an oligonucleotide is meant to refer to a naturally occurring oligonucleotide substantially similar, preferably having at least 60% homology, at least 70% homology, at least 80% homology, more preferably at least 90% homology, and most preferably at least 95% homology to either the entire oligonucleotide or a fragment thereof. An “analog” of an oligonucleotide can be without limitation a homologous molecule from the same species or from different species.

In addition to the part of the oligonucleotide sequence which is complementary to ACHE mRNA sequence, the antisense oligonucleotides of the invention may also comprise RNA sequences with enzymatic nucleolytic activity. Preferred nucleolytic sequences are ribozyme sequences which were shown to specifically interact with mRNA transcripts. Preferred ribozymes are hammerhead ribozymes. Another preferred ribozyme is the hairpin ribozyme structure, e.g., as derived from tobacco ringspot virus satellite RNA.

It is to be appreciated that the antisense oligonucleotides targeted to ACHE mRNA of the invention possess cross-species specificity and do not cause toxicity in rodents or primates.

In order to improve the penetration of the antisense oligonucleotides of the invention through the blood vessels, various modifications known in the art can be introduced. For example, the oligonucleotide molecule can be linked to a group comprising optionally partially unsaturated aliphatic hydrocarbon chain and/or one or more polar or charged groups such as a carboxylic acid group, an ester group, or an alcohol group. Alternatively, oligonucleotides can be linked to peptide structures, which are preferably membranotropic peptides. Such modified oligonucleotides penetrate membranes more easily, which is critical for their function and may therefore significantly enhance their activity. Palmityl-linked oligonucleotides have been described. Geraniol-linked oligonucleotides have also been described. Oligonucleotides linked to peptides, e.g., membranotropic peptides and their preparation have been described by Soukchareun et al. (Bioconjug. Chem. 9: 466-75, 1998). Modifications of antisense molecules that target the molecules to certain cells and enhance uptake of the oligonucleotide by said cells are described by Wang, J. (Controlled Release 53: 39-48, 1998).

The pharmaceutical composition to be administered comprises a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the antisense oligonucleotide is administered. Such pharmaceutical carriers can be sterile liquids, such as water or oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

The compositions can take the form of solutions, suspensions, emulsion, tablets, pills, capsules, powders, creams, sustained-release formulations and the like. The composition can be formulated as a suppository with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, and the like Examples of suitable pharmaceutical carriers are described in “Remington's Pharmaceutical Sciences” by E. W. Martin. Such compositions will contain a therapeutically effective amount of an antisense oligonucleotide together with a suitable amount of carrier so as to provide the form for proper administration to the subject.

The antisense oligonucleotides of the invention can also be enclosed within liposomes and thus be administered. The preparation and use of liposomes is well known in the art. For example, transfection reagents such as DOTAP (Roche Diagnostics), Lipofectin, Lipofectam, and Transfectam, which are available commercially, are used to enhance oligonucleotide uptake. Other methods of obtaining liposomes include the use of Sendai virus or of other viruses.

The amount of the antisense oligonucleotide which will be effective in treating inflammatory disorders in the gastrointestinal tract will depend on the nature of the disorder or condition, and can be determined by standard clinical techniques. The precise dose to be employed in the formulation will also depend on the route of administration, the seriousness of the disease or disorder and the clinical condition of the patient, and should be decided according to the judgment of the practitioner and each patient's circumstances.

Persons of ordinary skill in the art can easily estimate the dosage based on measured concentrations of the antisense oligonucleotide in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every month or year.

According to some embodiments, the pharmaceutical composition of the invention can be administered daily to a patient in need of such treatment at a dosage of the active ingredient between about 0.001 μg/g and about 50 μg/g. Preferably, the treatment and/or prevention comprises administering a dosage of the active ingredient of about 0.01 to about 5.0 μg/g. Most preferably, said dosage of active ingredient is of between about 0.05 to about 0.50 μg/g, and even most preferably, the dosage is from 0.15 to 0.50 μg/g of body weight of the patient.

Depending on the severity and responsiveness of the condition to be treated, dosing can be of a single or a plurality of administrations, with course of treatment lasting from several days to several weeks, or until diminution of the deterioration of the clinical state of the subject is achieved.

Methods of administration of the pharmaceutical composition comprising an antisense oligonucleotide targeted to ACHE mRNA include, but are not limited to, topical, intradermal, intramuscular, intraperitoneal, intravenous, subcutaneous, intranasal, epidural, ophthalmic, and oral routes. The composition can be administered by any convenient route, for example by infusion or bolus injection, by absorption through epithelial linings (e.g., oral mucosa, rectal or intestinal mucosa, transdermal, etc.), or can be administered together with other therapeutically active agents. According to a currently exemplary embodiment, the administration is by oral route. Pulmonary administration can also be employed, e.g., by use of an inhaler or nebulizer, and formulation with an aerosolizing agent.

It may be desirable to administer the pharmaceutical composition of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material. According to some preferred embodiments, administration can be by direct injection e.g., via a syringe, at the site of an inflammation.

For directed internal topical applications, the pharmaceutical composition can be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents.

For injection, the antisense oligonucleotide as an active ingredient of the pharmaceutical composition can be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The terms “therapeutically effective amount” of an antisense oligonucleotide targeted to AChE mRNA refers to that amount of the antisense oligonucleotide which is sufficient to provide a beneficial effect to the subject to which the antisense oligonucleotide is administered, namely an amount effective to ameliorate the symptoms associated with an inflammatory disorder such as inflammatory gastrointestinal disorder or prolong the survival of the subject being treated.

An antisense oligonucleotide targeted to ACHE mRNA can be tested in vivo for the desired therapeutic activity as well as for determination of a therapeutically effective dosage. For example, such oligonucleotides can be tested in suitable animal model systems prior to testing in humans, including, but not limited to, rats, mice, chicken, cows, monkeys, rabbits, and the like. For in vivo testing, prior to administration to humans, any animal model system known in the art can be used (see examples herein below).

A “therapeutic” activity is the activity of the antisense oligonucleotide that when administered to a subject who exhibits signs of pathology leads to the diminishing or eliminating those signs

Inflammatory disorders of the gastrointestinal tract which can be treated by the pharmaceutical composition of the invention include chronic inflammatory gastrointestinal disorders and acute inflammatory gastrointestinal disorders. Chronic inflammatory gastrointestinal disorders include, but are not limited to, Crohn's disease, inflammatory bowel disease, and ulcerative colitis. In other embodiments, the gastrointestinal disorder is associated with an immune function disorder. Examples of such immune function disorders include, but are not limited to, acquired immunodeficiency syndrome, chronic granulomatous disease, hypogammaglobulinemia, agammaglobulinemia, leukocyte adhesion deficiency, cyclic neutropenia, and glycogen storage disease 1b.

The efficacy of the treatment methods of the invention described herein is indicated by significant clinical improvement in treated patients. Clinical improvement means that any of a range of patient symptoms, biochemical indicators and pathological signs are ameliorated, eliminated or reduced, as assessed by methods known in the art. Patient symptoms include abdominal pain, rectal pain, chronic or intermittent diarrhea, weight loss, fever, rectal bleeding, tissue swelling and tenderness in the rectal area. Biochemical indicators include white blood cell count, sedimentation rate, red blood cell count, enzyme levels, protein levels, including C reactive protein, and body mineral concentrations. Visualization techniques used to assess pathological severity of gastrointestinal ulcers, abscesses, fissures and fistulae include x-ray, colonoscopy, sigmoidoscopy, computerized axial topography and video capsule endoscopy.

The antisense oligonucleotides directed against AChE can be administered alone or in conjunction with other therapeutic modalities. It is appropriate to administer the antisense oligonucleotides of the invention as part of a treatment regimen involving other therapies, such as drug therapy, which comprises, for example, immunosuppressive drugs.

### Example 1

### Effect of hEN101 on Inflammatory Bowel Diseases (IBD)

Colitis is a chronic inflammation of the bowel also known as Inflammatory Bowel Disease (IBD). This condition is characterized, at least in part, by an overproduction of pathological inflammatory cytokines such as TNF-α and IL-10. The current protocol employs the intra-rectal administration of 2,4,6-trinitrobenzene sulfonic acid (TNBS) to provoke severe colitis, which represents a well-validated model with many macroscopic and histologic similarities to IBD in human. Studies have indicated that TNBS-induced colitis responds favorably to many of the current therapies for IBD such as sulfasalazine or 5-aminosalacylic acid. In this study the effect of EN101 on colitis was studied.

BALB/C mice (6-8 week old male mice) were anesthetized (85% ketamine, 15% cellasine 2% solution; 30 μl IM/IP per mouse) for 90-120 min. TNBS, 150 mg/kg (dissolved in 40 μl of 0.9% NaCl and mixed with 40 μl of 50% ethanol) was administered by the intra-rectal route (via feeding needle connected to 1 ml syringe).

Nuclease resistant EN101 set forth in SEQ ID NO:2 was administered orally in a final volume of 200 μl of saline, once daily beginning either one day or two days after colitis induction.

Mice were provided ad libitum a commercial rodent diet (Harlan Teklad TRM Ra/Mouse Diet) and allowed free access to autoclaved water.

The treatments were as follows:

| Group No. | Animal No. | Induction | Treatment |
|---|---|---|---|
| 1 | 10 | TNBS | Saline (oral) |
| 2 | 10 | TNBS | Dexamethasone 100 μg |
| 3 | 10 | TNBS | EN101 25 μg/Kg in saline (oral) |
| 4 | 10 | TNBS | EN101 50 μg/Kg in saline (oral) |

Daily administration began at day 1 after colitis induction

| Group No. | Animal No. | Induction | Treatment |
|---|---|---|---|
| 5 | 10 | TNBS | Saline (oral) |
| 6 | 10 | TNBS | Dexamethasone 100 μg |
| 7 | 10 | TNBS | EN101 25 μg/Kg in saline (oral) |
| 8 | 10 | TNBS | EN101 50 μg/Kg in saline (oral) |
| 9 | 3 | | No treatment |

Daily administration began at day 2 after colitis induction.

Mice were inspected daily for their weight and signs of illness including diarrhea, rectal prolapse and rectal bleeding. The mice were sacrificed 7 days after TNBS administration

At the end of the study the colon was examined under a dissecting microscope (×5) to evaluate the macroscopic lesion according to the Wallace criteria. The Wallace score rates macroscopic colon lesions on a scale from 0 to 10 based on criteria reflecting inflammation such as hyperemia, thickening of the bowel and the extent of ulceration (Wallace, J. L., et al. 1989, Gastroenterology, 96:29).

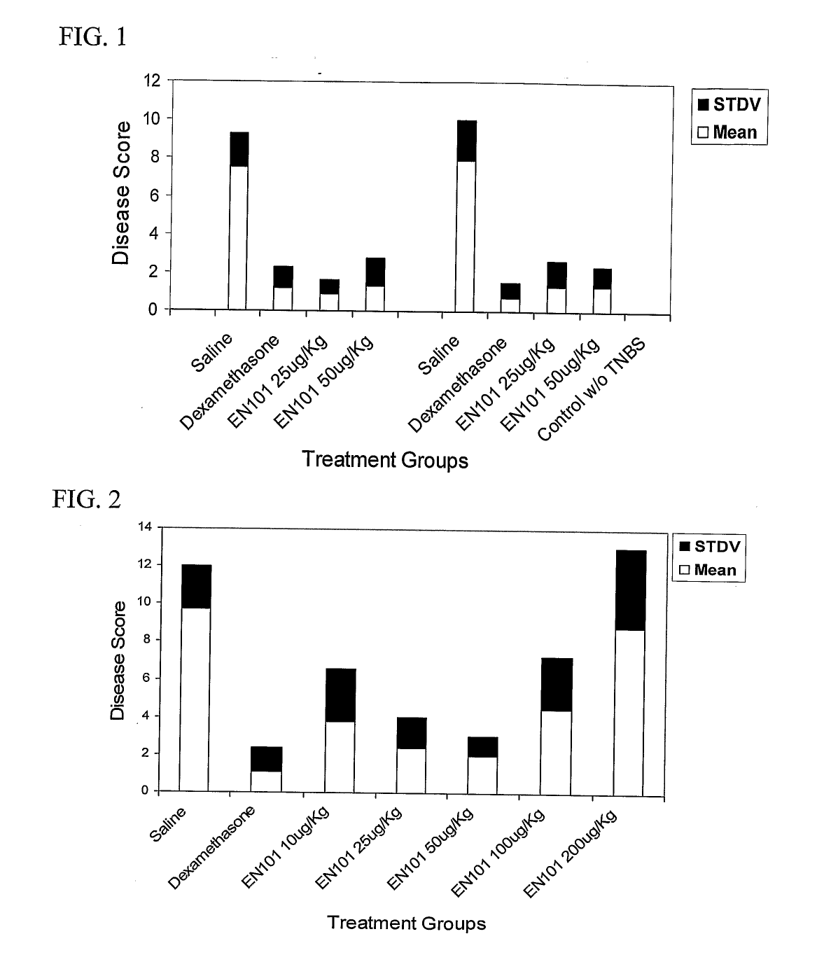

FIG. 1 shows the efficacy of nuclease resistant EN101 to treat IBD when administered orally either one day or two days after colitis induction. The efficacy of EN101 of SEQ ID NO:2 to alleviate colitis symptoms was prominent and comparable to that of Dexamethasone.

In another experiment, BALB/c mice were treated as follows:

| Group No. | Animal No. | Induction | Treatment |
|---|---|---|---|
| 1 | 10 | TNBS | Saline (oral) |
| 2 | 10 | TNBS | Dexamethasone 100 ug |
| 3 | 10 | TNBS | EN101 10 μg/Kg in saline (oral) |
| 4 | 10 | TNBS | EN101 25 μg/Kg in saline (oral) |
| 5 | 10 | TNBS | EN101 50 μg/Kg in saline (oral) |
| 6 | 10 | TNBS | EN101 100 μg/Kg in saline (oral) |
| 7 | 10 | TNBS | EN101 200 μg/Kg in saline (oral) |
| 8 | 3 | | No treatment |

Daily administration began 1 day before colitis induction.

FIG. 2 shows that oral daily administration of nuclease resistant EN101 of SEQ ID NO:2 beginning one day before colitis induction and for the next seven subsequent days protected the animals from the disease manifestation, specifically at low doses, i.e., 10 to 100 μg/Kg.

The effect of high doses of the nuclease resistant EN101 was next determined. The mice were treated as follows:

| Group No. | Animal No. | Induction | Treatment |
|---|---|---|---|
| 1 | 10 | TNBS | |
| 2 | 10 | TNBS | Saline (oral) |
| 3 | 10 | TNBS | Dexamethasone 100 ug |
| 4 | 10 | TNBS | EN101 100 μg/Kg in saline (oral) |
| 5 | 10 | TNBS | EN101 200 μg/Kg in saline (oral) |
| 6 | 10 | TNBS | EN101 500 μg/Kg in saline (oral) |
| 7 | 3 | | No treatment |

Daily administration began 1 day before colitis induction.

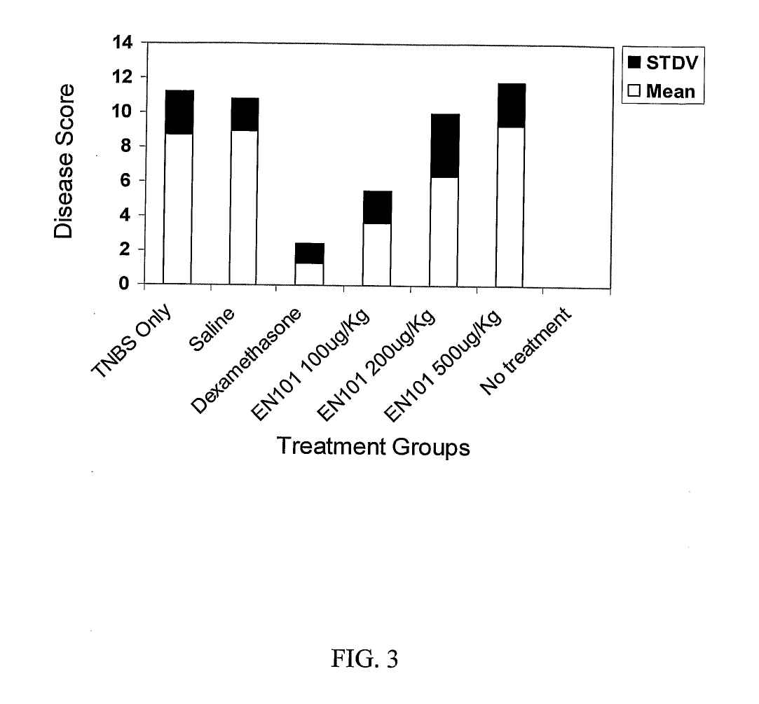

FIG. 3 shows that the nuclease resistant EN101 at 100 μg/Kg, when administered daily beginning one day before colitis induction and for the subsequent seven days, protected moderately colitis symptoms in the treated mice. However, higher doses of EN101, i.e., 200 or 500 μg/Kg, were less effective.

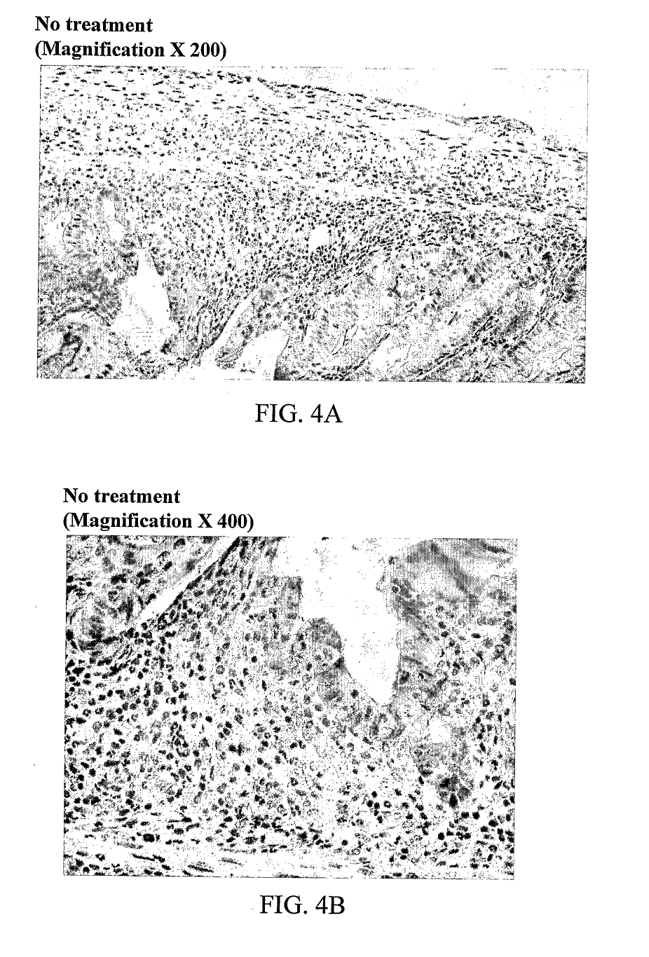

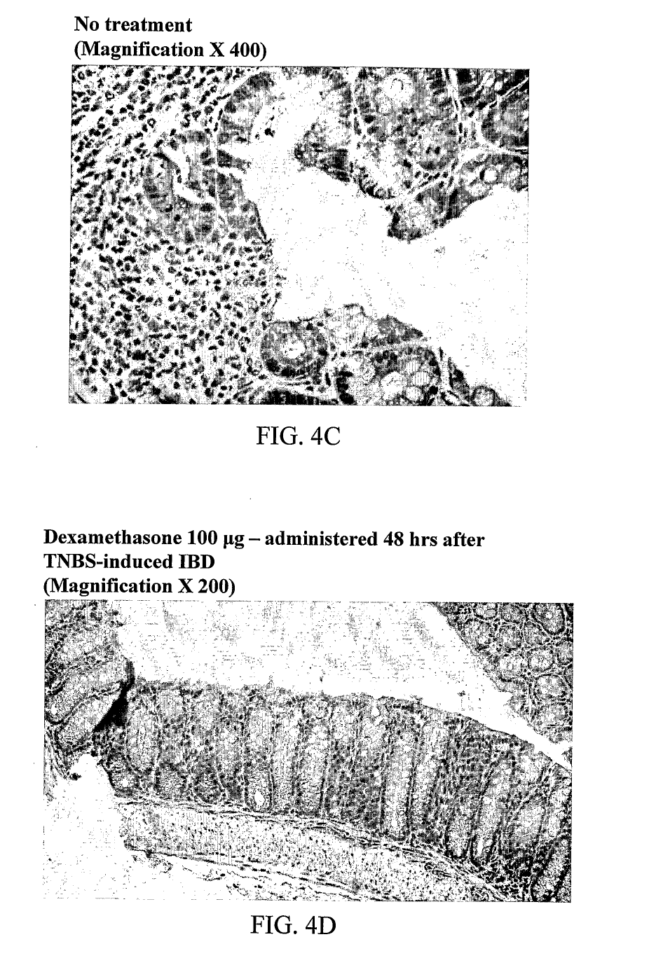

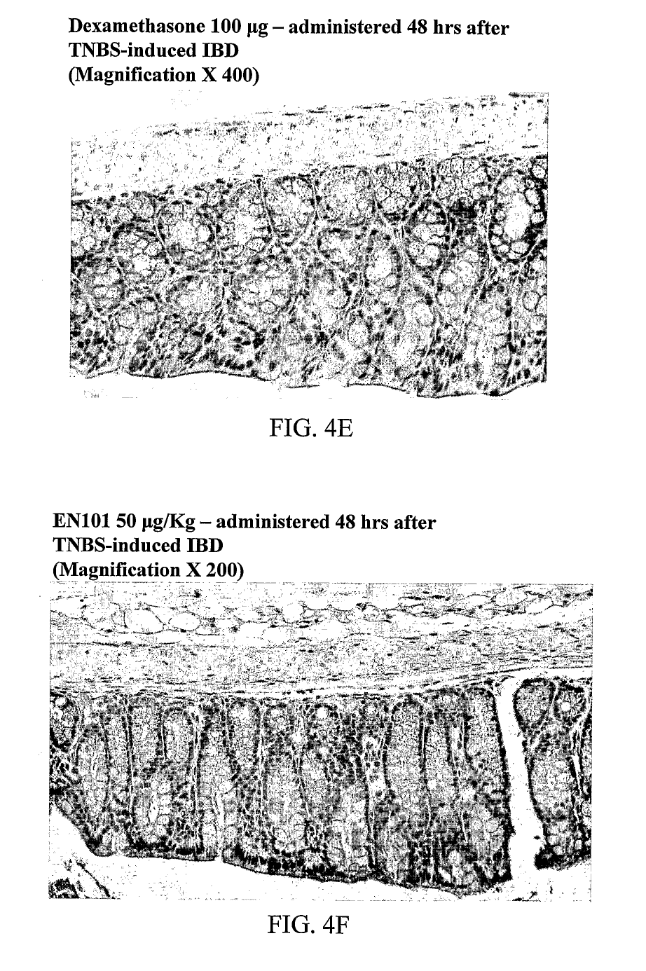

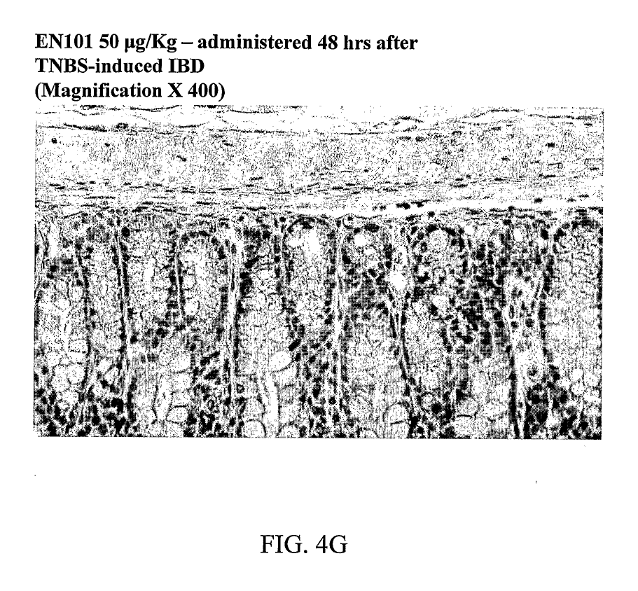

FIG. 4 shows photomicrographs of colon sections obtained from the above treated mice. As shown in FIGS. 4A-4C, the histology of the colon in TNBS-treated mice is characteristic of colitis, i.e., the tissue is disrupted, the villi structure is damaged, and the cells are granulated. FIGS. 4D and 4E show the effect of Dexamethasone on colon structure in TNBS-treated mice. Two days after colitis induction by TNBS in mice, Dexamethasone was administered daily at 100 μg. As shown in FIGS. 4D and 4E, Dexamethasone was capable of restoring the villi structure of the colon (FIGS. 4D and 4E). FIGS. 4F and 4G show the effect of EN101 on TNBS-induced colitis in mice. Two days after colitis induction by TNBS in mice, EN101 was administered daily at 50 μg/Kg. As shown in FIGS. 4F and 4G, the histology of the colon was similar to that of an intact colon, i.e., having a well organized villi structure. These results demonstrate that EN101 is highly efficient in treating IBD. It should be noted that EN101 is more advantageous than Dexamethasone as it is devoid of the steroidal adverse effects of Dexamethasone.

### Example 2

### Effect of EN101 on Ulcerative Colitis in Humans

Human subjects suffering from ulcerative colitis are treated with EN101 set forth in SEQ ID NO:2 at doses of 10, 25, 50 or 100 μg/Kg of body weight once daily for a period of eight weeks. Another group of subjects is treated with EN101 of SEQ ID NO:2 at doses of 10, 25, 50 or 100 μg/Kg of body weight given in divided doses twice daily for a period of eight weeks. After eight weeks of treatment the subjects are examined to evaluate their clinical condition.

### Example 3

### Effect of hEN101 on Endotoxin-Induced Uveitis (EIU)

Systemic injection of a sub-lethal dose of LPS induces bilateral acute ocular inflammation in susceptible strains of rats and mice. This endotoxin-induced uveitis (EIU) is an animal model for acute anterior uveitis in the human. In general, EIU peaks 24 hours after LPS injection and subsides within the next 96 hours. EIU is characterized by percolation of proteins from the serum and by infiltration of macrophages and neutrophils into the eye. In Lewis rats with EIU, acute inflammation develops mainly in the anterior chamber (iridocyclitis) and inflammatory cells may also infiltrate the vitreous and retina. In the mouse, the inflammation in the anterior chamber is less severe, and a relatively large number of neutrophils and macrophages accumulate in the vitreous, around the retinal vessels at the optic nerve head (posterior vitritis).

EIU is induced at day 0 in groups of five to eight male C57BL mice by a single subcutaneous injection of 0.2 mg Salmonella typhimurium LPS endotoxin (Difco Laboratories, Detroit, Mich.) in 0.05 mL PBS into the hind footpad. In mice treated with hEN101, the antisense oligonucleotide of SEQ ID NO:2 is administered orally or injected subcutaneously together with LPS. Control mice are injected with 0.05 mL PBS into the hind footpad. Mice are killed at 24±0.5 hours (day 1) or at 72±0.5 hours (day 3) after injection. All mice are maintained in an air-conditioned room with a 12-hour light/12-hour dark cycle and given free access to water and food until they are used for the experiments.

### Histopathology

Murine right eyes are enucleated and used for histopathology. Eyes are immersed in 4% glutaraldehyde for 30 minutes, fixed in 10% buffered formalin for at least 24 hours, and then embedded in methacrylate. Four- to 6-μm vertical sections are cut through the pupillary optic nerve axis and stained with hematoxylin and eosin (H&E). Infiltrating inflammatory cells in the anterior chamber and posterior vitreous are counted and identified histologically in a masked fashion by an ocular pathologist.

### Enzyme Linked Immunoabsorbent Assay (ELISA)

Serum samples are collected and pooled from each time point. IL-1α, IL-1β, 1L-6, IFN-γ, TNF-α, MIP-1α, and MIP-2 expression is determined by ELISA using commercially available kits (R&D Systems, Minneapolis, Minn.). These cytokines are tested because previous data indicate that they are induced in either animal models of anterior uveitis or in patients with acute anterior uveitis.

### Example 4

### Effect of hEN101 on the Survival and Cytokine Secretion of Activated Peritoneal Macrophages

The isolation of peritoneal macrophages was performed according to the method of Rossi et al., and Chino et al. (J. Leukoc. Biol. 78: 985-991, 2005; Int. Immunopharmacol. 5: 871-882, 2005). Briefly, C57BL6 mice were injected intraperitoneally with 0.5 gr/liter sterile thioglycolate (TG) medium (Novamed, Israel). Three days later peritoneal exudates cells (PECs) were collected by washing the peritoneum with cold phosphate-buffered saline (PBS). PECs were then seeded in 10-cm plates in RPMI1640 medium (GIBCO) supplemented with 10% fetal calf serum, 2 mM of L-glutamine, 100 units/ml penicillin and 100 mg/ml streptomycin. After 2 h at 37° C. in 5% CO2, the cells were washed twice with PBS to remove non-adherent cells. Adherent cells regarded as peritoneal macrophages (PMs) were incubated for 72 hr in complete medium at 37° C. in 5% CO2.

Thereafter, the PMs were seeded in a 12-well plate (Corning 10⁶ cells/well) and after a 4 h incubation, LPS (0.1 μg/ml), CpG sequence (TCC ATG ACG TTC CTG ACG TT set forth in SEQ ID NO:7; 1 and 10 μg/ml), non-CpG control sequence (TCC ATG AGC TTC CTG AGC TT set forth in SEQ ID NO:8; 1 and 10 μg/ml) and nuclease resistant EN101 of SEQ ID NO:2 (Avecia Limited; Lot: AMZ-01G-003-M) at concentrations of 0.1, 1, 10, and 100 μg/ml were added to the cells. The supernatants were collected after 24, 48, and 72 hr and the following cytokines: TNF-α (Elisa kit from R&D, Minneapolis, Minn.), IL-6 (Elisa kit from R&D, Minneapolis, Minn.), IL-1α (Elisa kit from PeproTech Asia), and the chemokine MIP-2 (Elisa kit from R&D, Minneapolis, Minn.), were measured by ELISA according to the manufacturer's instruction manuals. The viability and number of CD11b (MAC-1) cells in the culture were tested after 72 hr using FACS analysis. It is to be noted that PMs that express high levels of MAC-1, a marker that is characteristic of activated macrophages, and have high forward scatter (FSC) are defined as activated macrophages.

### Results

FACS analysis indicated that PMs, collected after 72 hr in culture, exhibited high FSC (FIG. 4A). Out of the total macrophages, 17.4% expressed high levels of MAC-1 (FIG. 4B).

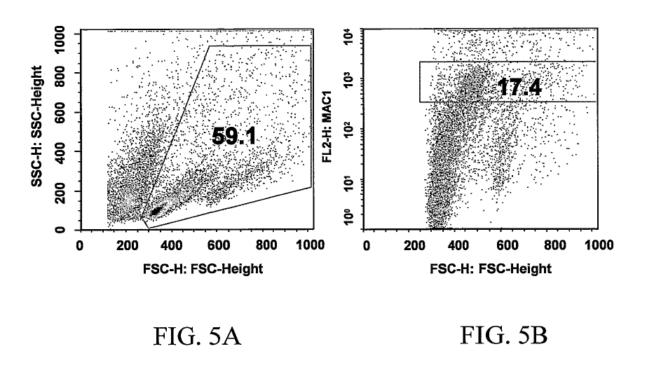

FIG. 5A shows the total number of PMs, and FIG. 5B shows the number of activated PMs, namely MAC1 (FIG. 5B). As can be seen in FIGS. 5A-B, CpG, non-CpG control, and LPS each increased the number of total and activated peritoneal macrophages; CpG exerted the highest activity, while LPS exerted the lowest activity. Surprisingly, EN101 at low concentrations inhibited the survival/proliferation of these cells.

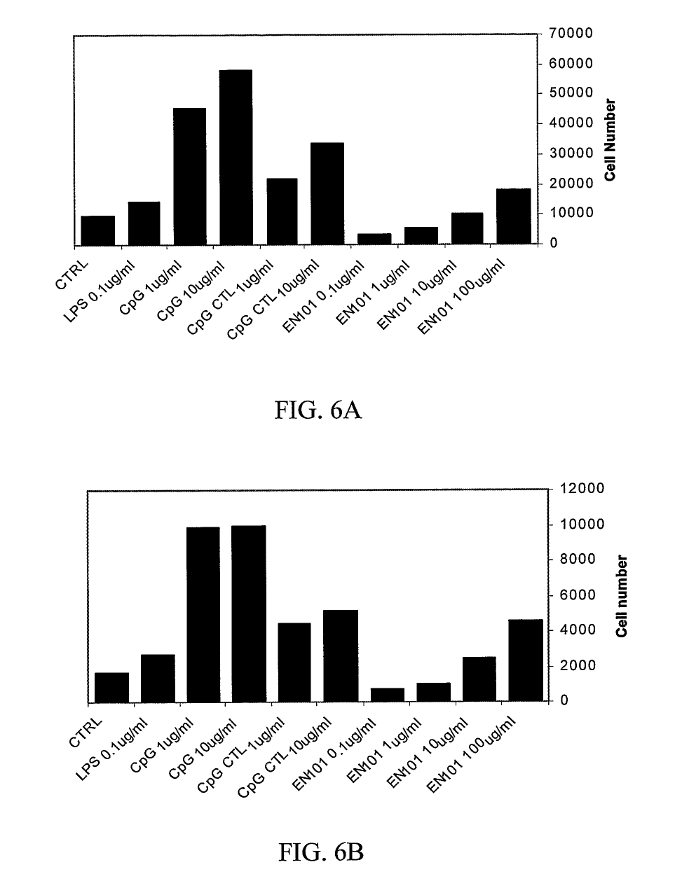

The levels of IL-6 (FIG. 6A), TNF-α (FIG. 6B), MIP-2 (FIG. 6C), and IL-1α (FIG. 6D) in the supernatants of activated PMs were tested by ELISA. As can be seen the levels of TNF-α, IL-6, and MIP-2 were strongly elevated by CpG and LPS and to a lower degree by the CpG control sequence. EN101 of SEQ ID NO:2 at low concentrations (0.1 and 1 μg/ml) did not induce secretion of IL-6, TNF-α and MIP-2, however, 100 μg/ml of EN101 induced moderate secretion of MIP-2. IL-1α was differentially regulated by all stimulators as compared to the other cytokines.

The present results demonstrate that EN101 affects the immune cells. As the Toll-like receptors (TLRs) are all connected to similar intracellular signaling pathways and activate similar cellular responses, including the production of pro-inflammatory cytokines such as IL-6, TNF- [symbol] and MIP-2 through the activation of the MyD88-dependent pathway, the question whether EN101 mediates its effects through TLR-MyD88-dependent pathway was next examined.

### Example 5

### Effect of hEN101 is Mediated Through TLR-My88 Signaling Pathway

The objective of this set of experiments was to test the role of TLR signaling pathway in EN101 activity. The technique was based on measuring the secretion of pro-inflammatory cytokines, particularly MIP-2, from peritoneal macrophages (PM) derived from wild type mice (C57BL6), TLR9 nock-out (KO) mice or myd88 KO mice (on C57BL6 background) following treatment with nuclease resistant EN101. MyD88⁻/⁻ in a C57BL6 background were originally prepared by Akira S at al., (Immunity, 9:143-150, 1998).

The isolation of peritoneal macrophages was performed according to the method of Rossi et al., and Chino et al. (J Leukoc. Biol. 78: 985-991, 2005; Int. Immunopharmacol. 5: 871-882, 2005). Briefly, C57BL6 or KO mice were injected intraperitoneally with 0.25 gr/l or 0.5 gr/liter sterile thioglycolate (TG) medium (Novamed, Israel). Three days later peritoneal exudate cells (PECs) were collected by washing the peritoneum with cold phosphate-buffered saline (PBS). PECs were then seeded in 10-cm plates in RPMI1640 medium (GIBCO) supplemented with 10% fetal calf serum, 2 mM of L-glutamine, 100 units/ml penicillin and 100 mg/ml streptomycin. After 2 h at 37° C. in 5% CO2, the cells were washed twice with PBS to remove non-adherent cells. Adherent cells regarded as peritoneal macrophages (PMs) were incubated for 72 hr in complete medium at 37° C. in 5% CO2.

Thereafter, the PMs were seeded in a 12-well plate (Corning 10⁶ cells/well) and after a 4 h incubation, LPS (0.1 μg/ml), CpG sequence (1 and 10 μg/ml), non-CpG control sequence (1 and 10 μg/ml) and EN101 of SEQ ID NO:2 (Avecia Limited; Lot: AMZ-01G-003-M) at concentrations of 0.1, 1, 10, and 100 μg/ml were added to the cells. The supernatants were collected after 24, 48, and 72 hr and the following cytokines: TNF-A (Elisa kit from R&D, Minneapolis, Minn.), IL-6 (Elisa kit from R&D, Minneapolis, Minn.), IL-1α (Elisa kit from PeproTech Asia), and the chemokine MIP-2 (Elisa kit from R&D, Minneapolis, Minn.), were measured by ELISA according to the manufacturer's instruction manuals. The viability and number of CD 11b (MAC-1) cells in the culture was tested after 72 hr using FACS analysis. PMs that express high levels of MAC-1 and have high forward scatter (FSC) are defined as activated macrophages.

### Results

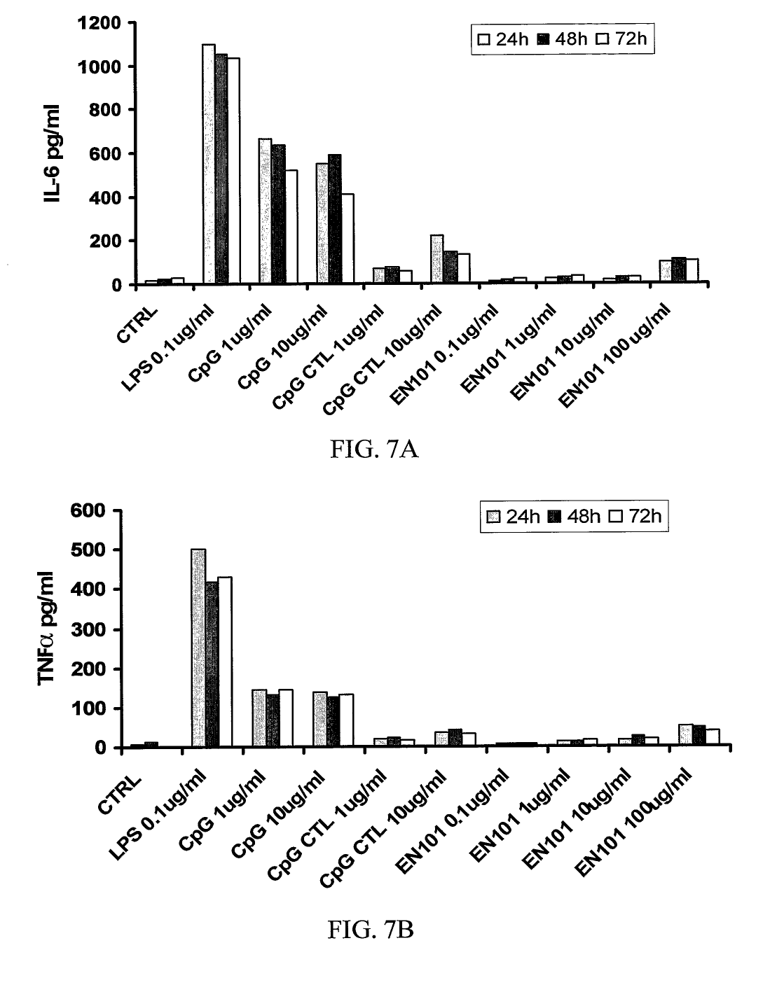

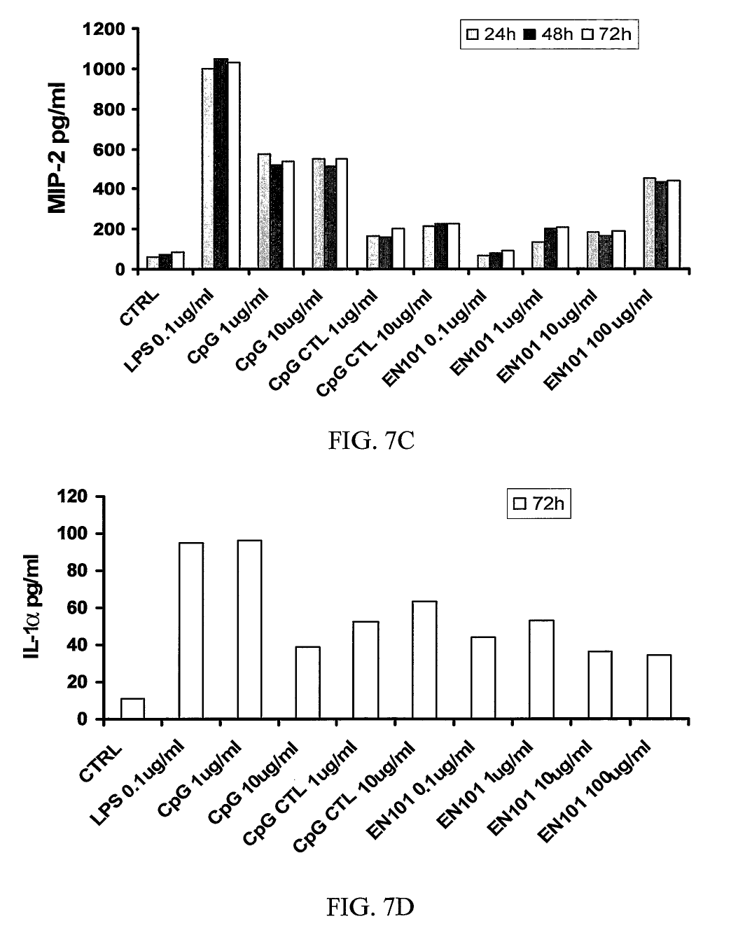

Total number of MAC-1 positive cells in WT mice (C57BL6) (FIG. 7A) or MyD88 KO mice (FIG. 7B) is presented. The number of MAC-1 positive cells was increased after treatment with LPS or CpG, while EN101 had a minor effect (FIG. 7A). In contrast, in Myd88 KO mice the number of MAC-1 positive cells following LPS, CpG, and EN101 treatment was reduced significantly.

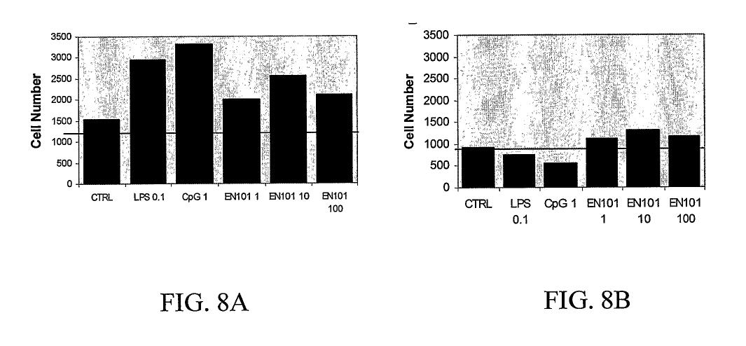

The levels of the chemokine MIP-2 in the supernatants of PMs derived from WT mice (C57BL6) (FIG. 8A-B) or MyD88 KO mice (FIG. 8C-D) were tested by ELISA. As can be seen in FIG. 8A, the levels of MIP-2 were elevated by CpG and LPS. In contrast, EN101 (1 μg/ml) inhibited MIP-2 secretion (FIG. 8B). The effect of CpG, LPS, and EN101 was almost totally inhibited in the MyD88 KO mice (FIG. 8C-D).

The present results demonstrate that LPS and CpG induce macrophage activation and elicit a significant MIP-2 secretion. The activation of macrophages and secretion of MIP-2 was shown to be dependent on MyD88 since MIP-2 secretion induced by CpG (FIG. 8) or by LPS (FIG. 8) was inhibited in MyD88 KO mice. In addition, the effect of EN101 on MIP-2 secretion was inhibited in the MyD88 KO, suggesting that the effect of EN101 on MIP-2 secretion is mediated by MyD88-TLR dependent pathway.

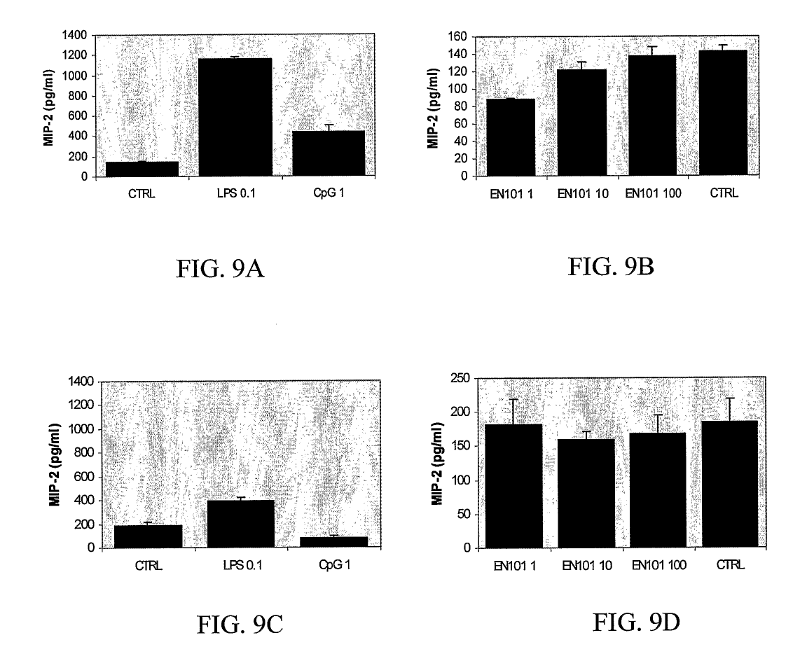

The total number of cells (FIGS. 9A-B) or MAC-1 positive cells (FIGS. 9C-D) in naïve WT (C57BL6) mice or TLR9 KO mice is shown. As shown in FIG. 9, the number of total cells or MAC-1 positive cells increased after treatment with CpG (FIGS. 9B, 9D). The nuclease resistant EN101 was ineffective in increasing macrophage cell number at low doses and had moderate effect at high doses (100 μg/ml). In contrast, in the TLR9 KO mice the number of total and MAC-1 positive cells was reduced significantly.

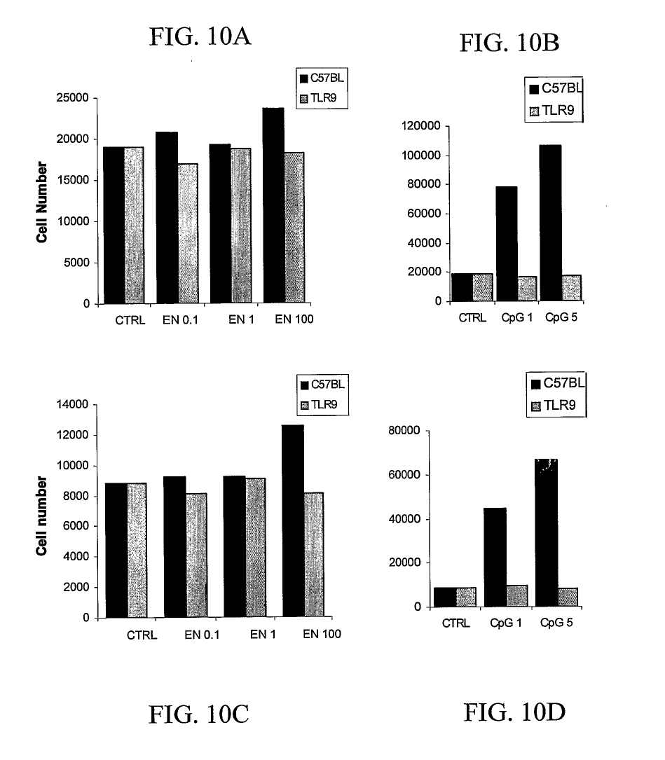

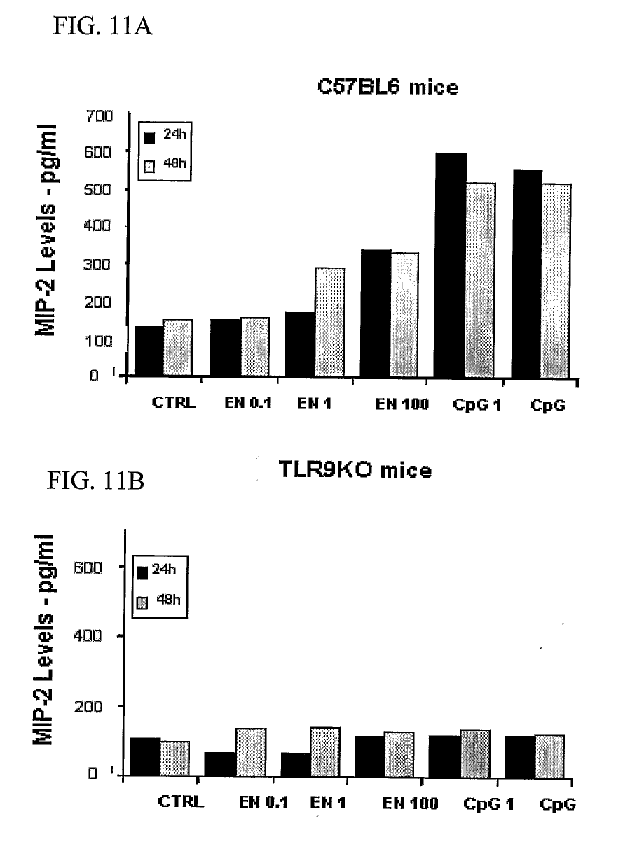

The levels of the chemokine MIP-2 in the supernatants of PM of WT (C57BL6) mice or TLR9 KO mice were next determined. As shown in FIG. 10, the levels of MIP-2 were strongly elevated by CpG (1, 5 μg/ml) or by EN101 of SEQ ID NO:2 in the control naïve C57BL6 mice, but not in the TLR9 KO mice.

Taken together the in-vivo and in-vitro results presented hereinabove, it should be emphasized that the present in vitro results provide partial insight into the mechanism of action of EN101 in treating IBD.

The present invention shows for the first time that EN101 can be used for treating inflammatory disorders other than those related to the central nervous system or peripheral nervous system which innervates voluntary muscles, such as IBD.

It will be appreciated by persons skilled in the art that the present invention is not limited by what has been particularly shown and described herein above. Rather the scope of the invention is defined by the claims that follow.

1. A method for treating an inflammatory disorder comprising administering to a subject in need thereof a therapeutically effective amount of a pharmaceutical composition comprising as an active agent an antisense oligonucleotide targeted to ACHE mRNA and a pharmaceutically acceptable carrier, wherein the inflammatory disorder is other than an inflammatory disorder of the central nervous system or the peripheral nervous system innervating voluntary muscles.

2. The method of claim 1, wherein the inflammatory disorder is an inflammatory gastrointestinal disorder.

3. The method of claim 2, wherein the inflammatory gastrointestinal disorder is selected from acute inflammatory gastrointestinal disorders and chronic inflammatory gastrointestinal disorders.

4. The method of claim 3, wherein the chronic inflammatory gastrointestinal disorder is inflammatory bowel disease.

5. The method of claim 4, wherein the inflammatory bowel disease is selected from the group consisting of Crohn's disease, Crohn's colitis, Crohn's enteritis, and ulcerative colitis.

6. The method of claim 4, wherein the inflammatory bowel disease is Crohn's disease.

7. The method of claim 2, wherein the inflammatory gastrointestinal disorder is due to a disorder selected from the group consisting of acquired immunodeficiency syndrome, chronic granulomatous disease, hypogammaglobulinemia, agammaglobulinemia, leukocyte adhesion deficiency, cyclic neutropenia, glycogen storage disease 1b, and celiac disease.

8. The method of claim 1, wherein the antisense oligonucleotide is an antisense oligodeoxynucleotide having the nucleotide sequence selected from the group consisting of SEQ ID NOs: 1, 3 to 5.

9. The method of claim 8, wherein the antisense oligodeoxynucleotide is nuclease resistant.

10. The method of claim 9, wherein at least one of the last nucleotides at the 3′ terminus of the antisense oligodeoxynucleotide is 2-O-methylated.

11. The method of claim 9, wherein three of the last nucleotides at the 3′ terminus of the antisense oligodeoxynucleotide are 2-O-methylated.

12. The method of claim 11, wherein the antisense oligodeoxynucleotide has a nucleotide sequence as set forth in SEQ ID NO:2.

13. The method of claim 9, wherein the antisense oligodeoxynucleotide comprises at least one phosphorothioate bond linking two nucleotide bases.

14. The method of claim 13, wherein the antisense oligodeoxynucleotide has a phosphorothioate bond linking the two last nucleotide bases at the 3′ terminus.

15. The method of claim 1, wherein administering the pharmaceutical composition is performed by oral, intravenous, intraarterial, intraperitoneal, subcutaneous, transdermal, intramuscular, intranasal, or inhalation administration route.

16. The method of claim 1, wherein administering the pharmaceutical composition is performed by oral administration route.

17. The method of claim 16, wherein administering the pharmaceutical composition is performed by daily administration.

18. The method of claim 17, wherein the antisense oligodeoxynucleotide is administered in a daily dose of 0.1 mg to 20 mg.
